(19) United States
(12) Patent Application Publication
(10) Pub. No.: US 2025/0019335 A1
(43) Pub. Date: Jan. 16, 2025

(54) NOVEL QUINIZARIN DERIVATIVE AND PREPARATION METHOD THEREOF

(71) Applicant: BENOBIO CO., LTD., Seongnam-si, Gyeonggi-do (KR)

(72) Inventor: In-Hyun LEE, Seoul (KR)

(21) Appl. No.: 18/711,951

(22) PCT Filed: Sep. 13, 2022
(86) PCT No.: PCT/KR2022/013624

§ 371 Date: May 21, 2024

(30) Foreign Application Priority Data
Nov. 26, 2021 (KR) 10-2021-0165050

(51) Int. Cl. C07C 49/83; A61K 31/122; A61K 31/337; A61K 31/357; A61P 1/16; C07D 305/08; C07D 319/14

(52) U.S. Cl. CPC C07C 49/83; A61K 31/122; A61K 31/337; A61K 31/357; A61P 1/16; C07D 305/08; C07D 319/14

(57) ABSTRACT

Proposed is a novel quinizarin derivative, and the novel quinizarin derivative has the abilities to inhibit a BET protein, and thus can be used as a pharmaceutical composition for the prevention or treatment of BET protein-related diseases, such as cancer, autoimmune or inflammatory diseases, metabolic diseases, and viral diseases. Particularly, the novel quinizarin derivative has the abilities to inhibit lipogenesis and the expression of liposynthetic factors, and thus can be used as a pharmaceutical composition for the treatment of non-alcoholic fatty liver.

### TECHNICAL FIELD

The present disclosure relates to a novel quinizarin derivative and a preparation method thereof.

### BACKGROUND ART

Post-translational modification (PTM) of histone is involved in the regulation of gene expression and chromatin organization in eukaryotic cells. Histone acetylation at specific lysine residues is PTM regulated by histone acetylase and histone deacetylase. The histone acetylation controls gene expression by mobilizing a protein complex in which a highly conserved protein called a bromodomain binds directly to acetylated lysine in histone and other proteins. There are more than 60 bromodomain-containing proteins in the human genome.

Among bromodomain-containing proteins, a bromodomain extra-terminal (BET) family includes BRD2, BRD3, BRD4, and BRDT, and except for BRDT, which is localized in the testis, the remaining proteins are widely expressed in various tissues. In addition, it has been reported that the BET protein family was associated with various diseases, including cancer, metabolic diseases, and inflammation.

For example, oncogenic fusions of BRD4 or BRD3, and nuclear protein in testis (NUT) genes, caused by chromosomal translocations, result in aggressive cancer named NUT midline carcinoma (French et al., J Clin Oncol, 22 (2004), 4135-9; French et al., J Clin Pathol, 63 (2008), 492-6). A BRD3/4 bromodomain is conserved in these fusion proteins, and a knockdown or selective BET bromodomain inhibitor, JQ1 causes the death of these cancer cells in both in vitro and an animal tumor model (Filippakopoulos et al., Nature, 468 (2010), 1067-73). It is known that JQ1 and other selective BET inhibitors bind to the BET bromodomain to prevent acetyl-lysine binding, which prevents BET proteins from interacting with chromatin, thereby preventing transcription from being regulated.

BRD4 was identified as a target in acute myeloid leukemia (AML) by RNAi screen (Zuber et al., Nature, 478 (2011), 524-8). These findings were validated using the BET inhibitors JQ1 and I-BET151 in vitro and in vivo (Dawson et al., Nature, 478 (2011), 529-33). In addition, it is also known that the BET inhibitors have broad anticancer activity in acute leukemia, multiple myeloma, and other hematologic malignancies. In several cancer models, acute downregulation of an oncogenic transcription factor Myc has been observed upon BET inhibition (Delmore et al., Cell, 146 (2011), 904-17; Mertz et al., Proc Natl Acad Sci USA, 108 (2011), 16669-74). Recent studies suggest that the BET inhibitors have the expandability to be applied to other carcinomas, including lung cancer, brain cancer, and the like.

It has been reported that another BET inhibitor I-BET762 closely related to JQ1 in its chemical structure and BET binding mode regulates the expression of key inflammatory genes in a mouse model and protects the human body from endotoxic shock and bacterial-induced sepsis (Nicodeme et al., Nature, 468 (2010), 1119-23). In addition, these results have been used to support clinical evaluation of a BET inhibitor RVX-208 in clinical trials for patients with atherosclerosis, coronary artery disease, dyslipidemia, diabetes, and other cardiovascular diseases (McNeill, Curr Opin Investig Drugs, 3 (2010), 357-64 and www.clinicaltrials.gov).

It was found that both RVX-208 and I-BET762 upregulated apolipoprotein A-I, which is important in reducing tissue levels of cholesterol. In addition, it is considered that BET proteins are involved in the proliferation and transcription regulation of several viruses, so that the BET inhibitors may have antiviral activity (Weidner-Glunde, Frontiers in Bioscience 15 (2010), 537-549).

Although several bromodomain inhibitors are known in clinical and preclinical use, there is an urgent need for the development of new bromodomain inhibitors that can solve the problems of recurrence of diseases and resistance to therapeutic agents and reduce side effects.

Meanwhile, fatty liver is a pathological condition in which excessive neutral fat accumulates in liver cells, and is medically defined as a condition in which neutral fat accounts for 5% or more of the liver weight. The fatty liver is classified into alcoholic and non-alcoholic fatty liver depending on whether it is caused by excessive alcohol consumption. The non-alcoholic fatty liver disease (NAFLD) is a group of diseases that encompass all aspects of the diseases from non-alcoholic fatty liver disease to steatohepatitis and cirrhosis. Simple non-alcoholic fatty liver disease is a disease in which there is only fat deposition in the liver, but there are no hepatocyte damage and fibrosis findings, and only high fat deposition in the liver tissue increases due to causes such as insulin resistance. Non-alcoholic fatty liver disease may progress to non-alcoholic steatohepatitis (NASH), which is accompanied by fibrosis, due to liver cell damage caused by inflammatory responses due to oxidative stress, etc.

Unlike simple fatty liver, non-alcoholic steatohepatitis may progress to cirrhosis and liver cancer, causing irreversible liver damage, and increases mortality and overall mortality associated with liver disease (Hepatology, 2012 (55): 2005-2023). For this reason, according to US statistics, as of 2013, non-alcoholic steatohepatitis is the second most common cause of liver transplantation after hepatitis C, and it is expected that after 2020, non-alcoholic steatohepatitis will be the first cause of liver transplantation ahead of hepatitis C, so that the development of drugs to prevent and treat the disease is urgently needed (Clinical Gastroenterology and Hepatology, 2019 (17): 748-755).

Korean Patent Registration No. 10-1903150 is a patent for a composition for preventing or treating obesity including quinizarin as an active ingredient. Korean Patent Registration

10-1903150 discloses a health functional food composition for preventing or improving obesity caused by neutral fat characterized by reducing mRNA or protein expression levels of at least one selected from the group consisting of CCAAT/enhancer binding protein peroxisome-α (C/EBP-α), CCAAT/enhancer binding protein peroxisome-β (C/EBP-β), Peroxisome proliferator-activated receptor-γ (PPAR-γ) and adipocyte protein 2 (aP2) using the composition including quinizarin as an active ingredient, but the novel quinizarin derivative according to the present disclosure is not mentioned.

### DETAILED DESCRIPTION OF THE INVENTION

### Technical Problem

An object to be achieved by the present disclosure is to provide a novel quinizarin derivative.

Another object to be achieved by the present disclosure is to provide a method for preparing the quinizarin derivative.

Yet another object to be achieved by the present disclosure is to provide a bromodomain extra-terminal (BET) protein inhibitor including the quinizarin derivative.

Yet another object to be achieved by the present disclosure is to provide a composition for treating non-alcoholic fatty liver including the quinizarin derivative.

Objects of the present disclosure are not limited to the above-mentioned objects, and other objects, which are not mentioned above, can be clearly understood by those skilled in the art from the following descriptions.

### Technical Solution

According to a first aspect of the present disclosure, there is provided a novel quinizarin derivative, including a compound represented by the following Chemical Formula 1 or 2:

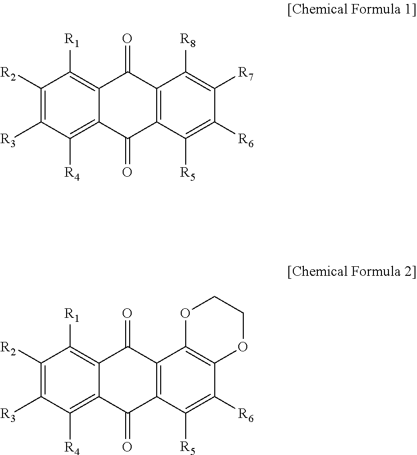

(in Chemical Formula 1 or 2,
R₁, R₂, R₃, R₄, R₅, R₆, R₇ and R₅ are each independently hydrogen, hydroxy, linear or branched C₁-C₁₂ alkyl which may be substituted, C₃-C₁₂ cycloalkyl which may be substituted, C₆-C₁₈ aryl which may be substituted, or alkoxy, and
the substitution is performed by oxygen, nitrogen, sulfur, hydroxyl, linear or branched C₁-C₆ alkyl, C₆-C₂₀ aryl, halogen, alkoxy, ether or a combination thereof).

According to an embodiment of the present disclosure, the quinizarin derivative may include any one of the following compounds, but is not limited thereto:

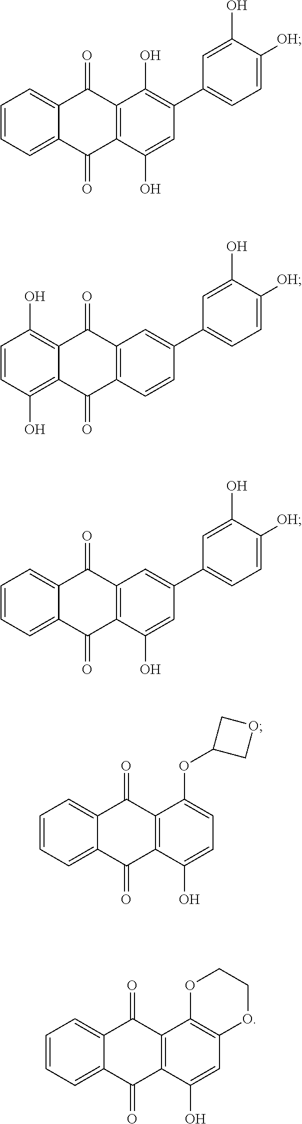 [SMILES: O=C1c2ccccc2C(=O)c2c(O)c(-c3ccc(O)c(O)c3)cc(O)c21 ; O=C1c2ccc(-c3ccc(O)c(O)c3)cc2C(=O)c2c(O)ccc(O)c21 ; O=C1c2ccccc2C(=O)c2c(O)cc(-c3ccc(O)c(O)c3)cc21 ; O=C1c2ccccc2C(=O)c2c(OC3COC3)ccc(O)c21 ; O=C1c2ccccc2C(=O)c2c3c(cc(O)c21)OCCO3]

According to a second aspect of the present disclosure, there is provided a method for preparing a novel quinizarin derivative according to claim 1, including reacting an anthracene derivative with a compound represented by Chemical Formula 3, 4, or 5 below:

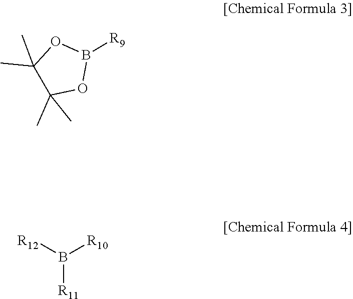

Br—R₁₃ [Chemical Formula 5]

(in Chemical Formulas 3 to 5,
R₉, R₁₀, R₁₁, R₁₂, and R₁₃ are each independently hydrogen, hydroxy, linear or branched C₁-C₁₂ alkyl which may be substituted, C₃-C₁₂ cycloalkyl which may be substituted, C₆-C₁₈ aryl which may be substituted, or alkoxy, and
the substitution is performed by oxygen, nitrogen, sulfur, hydroxyl, linear or branched C₁-C₆ alkyl, C₆-C₂₀ aryl, halogen, alkoxy, ether or a combination thereof).

According to an embodiment of the present disclosure, the anthracene derivative may be selected from the group consisting of 2-bromo-1,4-dihydroxy-anthracene-9,10-dione, 6-bromo-1,4-dihydroxy-anthracene-9,10-dione, 3-bromo-1-hydroxy-anthracene-9,10-dione, 1,4-dihydroxy-anthracene-9,10-dione, 1,2,4-trihydroxy-anthracene-9,10-dione, and combinations thereof, but is not limited thereto.

According to an embodiment of the present disclosure, the compound represented by Chemical Formula 3 may include 2-(3,4-dimethoxyphenyl)-4,4,5,5-tetramethyl-1,3,2-dioxaborolane, or 4,4,5,5-tetramethyl-2-(4,4,5,5-tetramethyl-1,3,2-dioxaborolan-2-yl)-1,3,2-dioxaborolane, but is not limited thereto.

According to an embodiment of the present disclosure, the compound represented by Chemical Formula 4 may include 3,4-dimethoxyphenylboronic acid, but is not limited thereto.

According to an embodiment of the present disclosure, the compound represented by Chemical Formula 5 may include benzyl bromide or 2-bromoethanol, but is not limited thereto.

According to an embodiment of the present disclosure, the quinizarin derivative may include any one of the following compounds, but is not limited thereto:

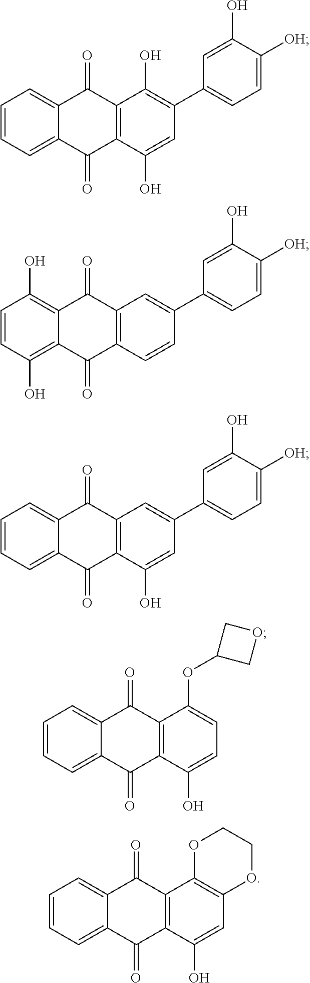

According to a third aspect of the present disclosure, there is provided a bromodomain extra-terminal (BET) protein inhibitor including the novel quinizarin derivative according to the first aspect of the present disclosure.

According to a fourth aspect of the present disclosure, there is provided a composition for treating non-alcoholic fatty liver including the novel quinizarin derivative according to the first aspect of the present disclosure.

The above-mentioned technical solutions are merely exemplary and should not be construed as limiting the present disclosure. In addition to the above-described embodiments, additional embodiments may exist in the drawings and detailed description of the disclosure.

### Advantageous Effects of the Invention

According to the present disclosure, the novel quinizarin derivative has the abilities to inhibit a bromodomain extra-terminal (BET) protein, and thus can be used as a pharmaceutical composition for the prevention or treatment of BET protein-related diseases, such as cancer, autoimmune or inflammatory diseases, metabolic diseases, and viral diseases.

Particularly, the novel quinizarin derivative has the abilities to inhibit lipogenesis and the expression of liposynthetic factors, and thus can be used as a pharmaceutical composition for the treatment of non-alcoholic fatty liver.

However, effects obtainable herein are not limited to the effects described above, and other effects may be present.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a hydrogen atom nuclear magnetic resonance (1H NMR) spectrum of a quinizarin derivative prepared in Example 1 of the present disclosure;

FIG. 2 is an LC-MS result of the quinizarin derivative prepared in Example 1 of the present disclosure;

FIG. 3 is a hydrogen atom nuclear magnetic resonance (1H NMR) spectrum of a quinizarin derivative prepared in Example 2 of the present disclosure;

FIG. 4 is an LC-MS result of the quinizarin derivative prepared in Example 2 of the present disclosure;

FIG. 5 is a hydrogen atom nuclear magnetic resonance (1H NMR) spectrum of a quinizarin derivative prepared in Example 3 of the present disclosure;

FIG. 6 is an LC-MS result of the quinizarin derivative prepared in Example 3 of the present disclosure;

FIG. 7 is a hydrogen atom nuclear magnetic resonance (1H NMR) spectrum of a quinizarin derivative prepared in Example 4 of the present disclosure;

FIG. 8 is an LC-MS result of the quinizarin derivative prepared in Example 4 of the present disclosure;

FIG. 9 is a hydrogen atom nuclear magnetic resonance (1H NMR) spectrum of a quinizarin derivative prepared in Example 5 of the present disclosure;

FIG. 10 is an LC-MS result of the quinizarin derivative prepared in Example 5 of the present disclosure;

FIG. 11 illustrates results of measuring ability to inhibit lipogenesis of a quinizarin derivative according to an embodiment of the present disclosure;

FIG. 12 is a result of RT-PCT reaction to confirm ability to inhibit expression of liposynthesis factors of a quinizarin derivative according to an embodiment of the present disclosure;

FIG. 13 illustrates a result of confirming SREBP-1 expression after treating fatty liver-induced cells with a quinizarin derivative at each concentration according to an embodiment of the present disclosure; and

FIG. 14 illustrates a result of confirming FAS expression after treating fatty liver-induced cells with a quinizarin derivative at each concentration according to an embodiment of the present disclosure.

### BEST MODE FOR CARRYING OUT THE INVENTION

Hereinafter, embodiments of the present disclosure will be described in detail so as to be easily implemented by those skilled in the art, with reference to the accompanying drawings. However, the present disclosure may be embodied in many different forms and is not limited to the embodiments to be described herein. In addition, parts not related to the description have been omitted in order to clearly describe the present disclosure in the drawings and throughout the present specification, like reference numerals designate like elements.

Further, throughout this specification, when a certain part is “connected” with the other part, it is meant that the certain part may be “directly connected” with the other part and “electrically connected” with the other part with another element interposed therebetween.

Throughout the present specification, it will be understood that when a certain member is located “on”, “above”, “at the top of”, “under”, “below”, and “at the bottom of” the other member, a certain member is in contact with the other member and another member may also be present between the two members.

Throughout the specification, a case where a part “includes” an element will be understood to imply the inclusion of stated elements but not the exclusion of any other elements unless explicitly described to the contrary.

The terms “about”, “substantially”, and the like to be used in the present specification are used as a numerical value or a value close to the numerical value when inherent manufacturing and material tolerances are presented in the stated meaning, and used to prevent an unscrupulous infringer from unfairly using disclosed contents in which precise or absolute numerical values are mentioned to help in the understanding of the present disclosure. Throughout the present specification, the term of “step to” or “step of” does not mean “step for”.

Throughout the present specification, the term “combinations thereof” included in the expression of the Markush form means one or more mixtures or combinations selected from the group consisting of components described in the expression of the Markush form and means to include at least one selected from the group consisting of the components.

Throughout the present specification, “A and/or B” means “A or B, or A and B”.

Hereinafter, a novel quinizarin derivative of the present disclosure and a preparation method thereof will be described in detail with reference to embodiments, Examples, and drawings. However, the present disclosure is not limited to these embodiments, Examples, and drawings.

According to a first aspect of the present disclosure, there is provided a novel quinizarin derivative, including a compound represented by the following Chemical Formula 1 or 2:

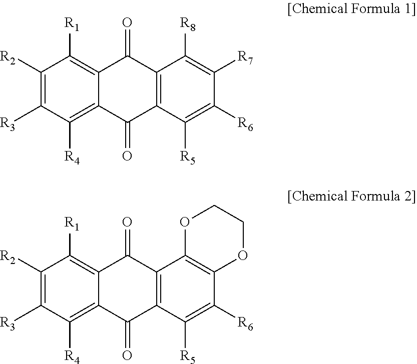

(in Chemical Formulas 1 and 2,
R₁, R₂, R₃, R₄, R₅, R₆, R₇ and R⁸ are each independently hydrogen, hydroxy, linear or branched C₁-C₁₂ alkyl which may be substituted, C₃-C₁₂ cycloalkyl which may be substituted, C₆-C₁₈ aryl which may be substituted, or alkoxy, and
the substitution is performed by oxygen, nitrogen, sulfur, hydroxyl, linear or branched C₁-C₆ alkyl, C₆-C₂₀ aryl, halogen, alkoxy, ether or a combination thereof).

According to the present disclosure, the novel quinizarin derivative has the abilities to inhibit a bromodomain extra-terminal (BET) protein, and thus can be used as a pharmaceutical composition for the prevention or treatment of BET protein-related diseases, such as cancer, autoimmune or inflammatory diseases, metabolic diseases, and viral diseases.

Particularly, according to the present disclosure, the novel quinizarin derivative has the abilities to inhibit lipogenesis and the expression of liposynthetic factors, and thus can be used as a pharmaceutical composition for the treatment of non-alcoholic fatty liver.

According to an embodiment of the present disclosure, the quinizarin derivative may include any one of the following compounds, but is not limited thereto:

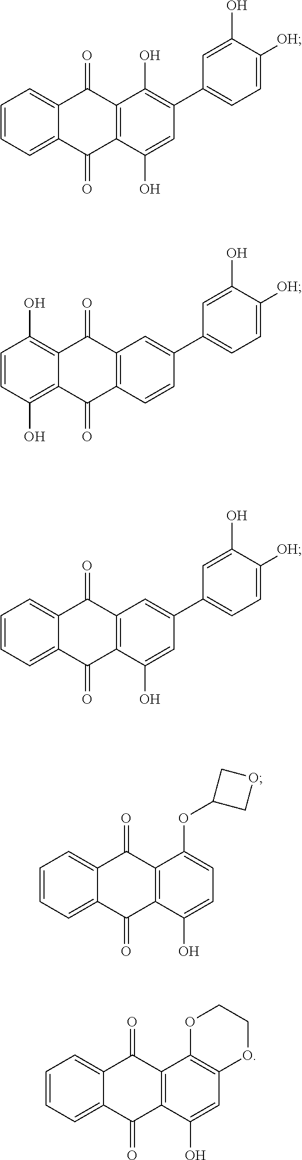 [SMILES: O=C1c2ccccc2C(=O)c2c(O)c(-c3ccc(O)c(O)c3)cc(O)c21 ; O=C1c2ccc(-c3ccc(O)c(O)c3)cc2C(=O)c2c(O)ccc(O)c21 ; O=C1c2ccccc2C(=O)c2c(O)cc(-c3ccc(O)c(O)c3)cc21 ; O=C1c2ccccc2C(=O)c2c(OC3COC3)ccc(O)c21 ; O=C1c2ccccc2C(=O)c2c3c(cc(O)c21)OCCO3]

According to a second aspect of the present disclosure, there is provided a method for preparing a novel quinizarin derivative according to claim 1, including reacting an anthracene derivative with a compound represented by Chemical Formula 3, 4, or 5 below:

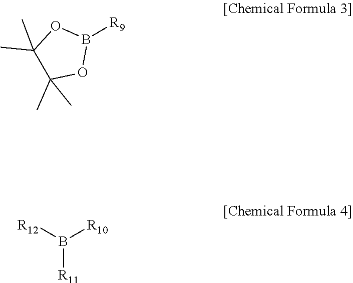

Br—R₁₃ [Chemical Formula 5]

(in Chemical Formulas 3 to 5,
R₉, R₁₀, R₁₁, R₁₂, and R₁₃ are each independently hydrogen, hydroxy, linear or branched C₁-C₁₂ alkyl which may be substituted, C₃-C₁₂ cycloalkyl which may be substituted, C₆-C₁₈ aryl which may be substituted, or alkoxy, and
the substitution is performed by oxygen, nitrogen, sulfur, hydroxyl, linear or branched C₁-C₆ alkyl, C₆-C₂₀ aryl, halogen, alkoxy, ether or a combination thereof).

With respect to the method for preparing the novel quinizarin derivative according to the second aspect of the present disclosure, the detailed description of parts duplicated with the first aspect of the present disclosure has been omitted, but even if the description has been omitted, the contents disclosed in the first aspect of the present disclosure may be equally applied to the second aspect of the present disclosure.

According to an embodiment of the present disclosure, the anthracene derivative may be selected from the group consisting of 2-bromo-1,4-dihydroxy-anthracene-9,10-dione, 6-bromo-1,4-dihydroxy-anthracene-9,10-dione, 3-bromo-1-hydroxy-anthracene-9,10-dione, 1,4-dihydroxy-anthracene-9,10-dione, 1,2,4-trihydroxy-anthracene-9,10-dione, and combinations thereof, but is not limited thereto.

According to an embodiment of the present disclosure, the compound represented by Chemical Formula 3 may include 2-(3,4-dimethoxyphenyl)-4,4,5,5-tetramethyl-1,3,2-dioxaborolane, or 4,4,5,5-tetramethyl-2-(4,4,5,5-tetramethyl-1,3,2-dioxaborolan-2-yl)-1,3,2-dioxaborolane, but is not limited thereto.

According to an embodiment of the present disclosure, the compound represented by Chemical Formula 4 may include 3,4-dimethoxyphenylboronic acid, but is not limited thereto.

According to an embodiment of the present disclosure, the compound represented by Chemical Formula 5 may include benzyl bromide or 2-bromoethanol, but is not limited thereto.

The compound prepared by reacting the anthracene derivative and the compound represented by Chemical Formula 3, 4, or 5 may have a functional group substituted through an additional process.

For example, the compound produced by reacting the anthracene derivative, 2-bromo-1,4-dihydroxy-anthracene-9,10-dione with the compound represented by Chemical Formula 3, 2-(3,4-dimethoxyphenyl)-4,4,5,5-tetramethyl-1,3,2-dioxaborolane may further react with boron tribromide and dichloroethane to substitute a functional group, but is not limited thereto.

According to an embodiment of the present disclosure, the quinizarin derivative may include any one of the following compounds, but is not limited thereto:

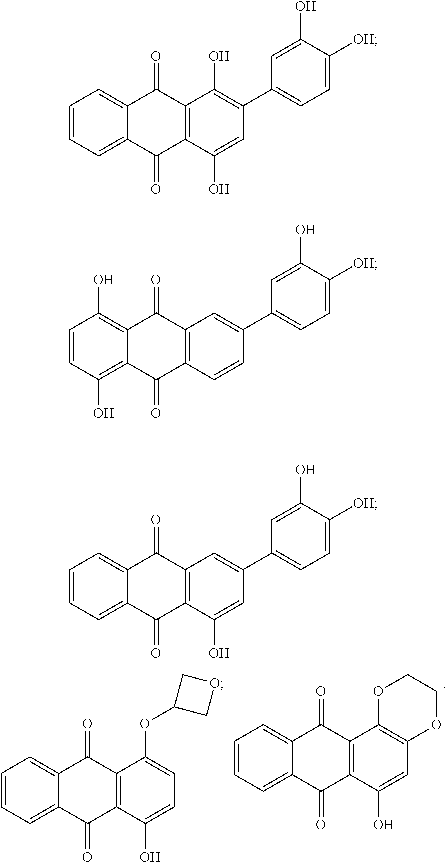

According to a third aspect of the present disclosure, there is provided a bromodomain extra-terminal (BET) protein inhibitor including the novel quinizarin derivative according to the first aspect of the present disclosure.

With respect to the BET protein inhibitor according to the third aspect of the present disclosure, the detailed description of the duplicated parts with the first aspect and/or the second aspect of the present disclosure has been omitted, but even if the description thereof has been omitted, the contents disclosed in the first aspect and/or the second aspect of the present disclosure may be equally applied to the third aspect of the present disclosure.

According to the present disclosure, the novel quinizarin derivative has the abilities to inhibit a BET protein, and thus can be used as a pharmaceutical composition for the prevention or treatment of BET protein-related diseases, such as cancer, autoimmune or inflammatory diseases, metabolic diseases, and viral diseases.

According to a fourth aspect of the present disclosure, there is provided a composition for treating non-alcoholic fatty liver including the novel quinizarin derivative according to the first aspect of the present disclosure.

With respect to the composition for treating non-alcoholic fatty liver according to the fourth aspect of the present disclosure, the detailed description of the duplicated parts with the first aspect and/or the second aspect of the present disclosure has been omitted, but even if the description thereof has been omitted, the contents disclosed in the first aspect and/or the second aspect of the present disclosure may be equally applied to the fourth aspect of the present disclosure.

The novel quinizarin derivative according to the present disclosure has the abilities to inhibit lipogenesis and the expression of liposynthetic factors, and thus can be used as a pharmaceutical composition for the treatment of non-alcoholic fatty liver.

Hereinafter, the present disclosure will be described in more detail with reference to the following Examples, but the following Examples are only for illustrative purposes and are not intended to limit the scope of the present disclosure.

### Example 1

A novel quinizarin derivative according to the first aspect of the present disclosure was synthesized according to Reaction Formula 1 below.

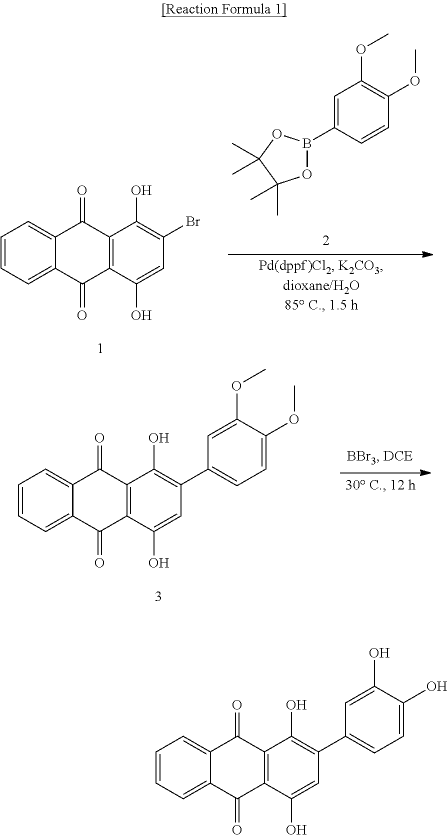

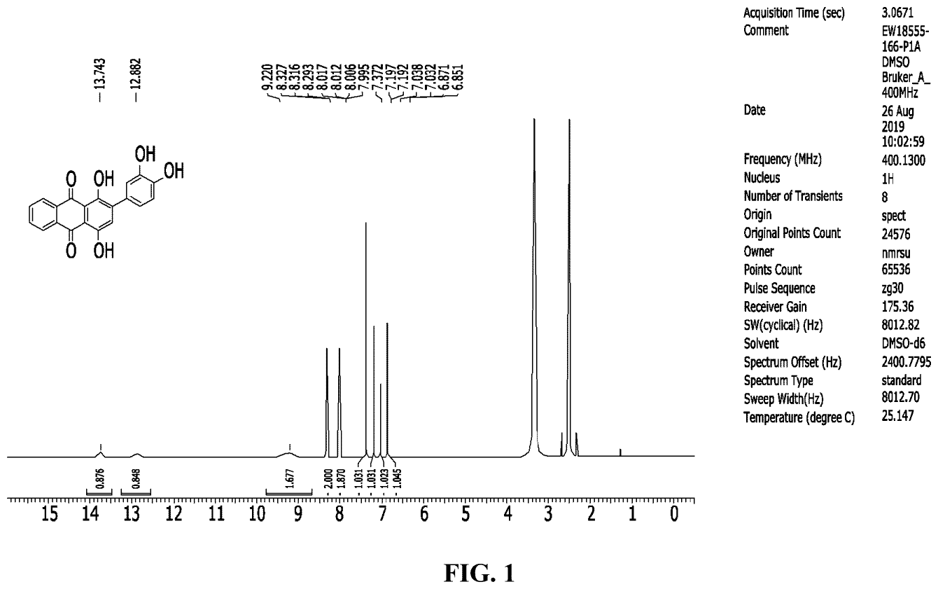

FIG. 1 is a hydrogen atom nuclear magnetic resonance (1H NMR) spectrum of a quinizarin derivative prepared in Example 1 of the present disclosure.

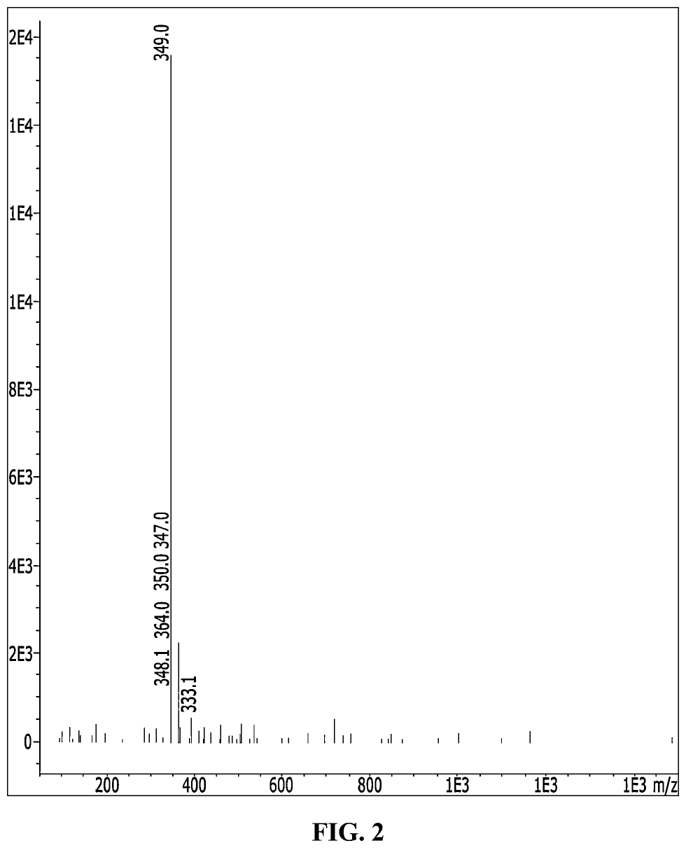

FIG. 2 is an LC-MS result of the quinizarin derivative prepared in Example 1 of the present disclosure.

Through FIGS. 1 and 2, it was confirmed that Compound 1 below was actually prepared.

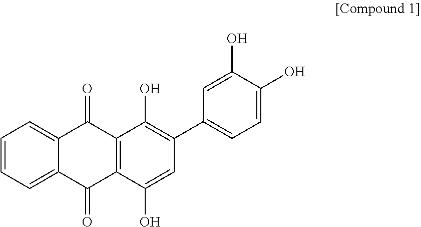 [SMILES: O=C1c2ccccc2C(=O)c2c(O)c(-c3ccc(O)c(O)c3)cc(O)c21]

### Example 2

A novel quinizarin derivative according to the first aspect of the present disclosure was synthesized according to Reaction Formula 2 below.

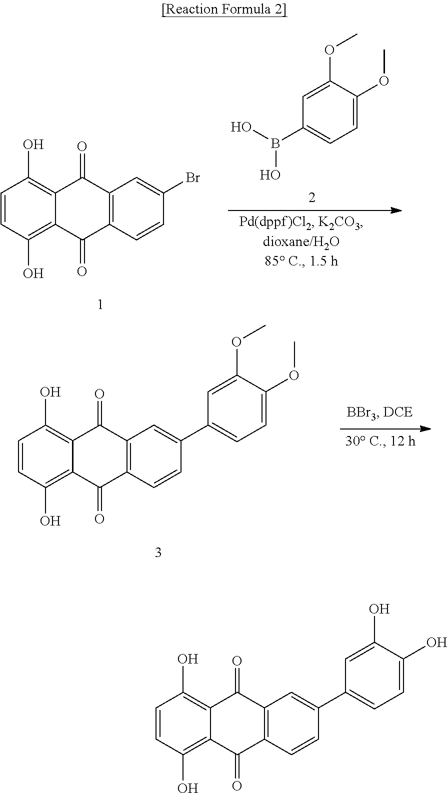 [SMILES: O=C1c2ccc(Br)cc2C(=O)c2c(O)ccc(O)c21 ; COc1ccc(B(O)O)cc1OC ; COc1ccc(-c2ccc3c(c2)C(=O)c2c(O)ccc(O)c2C3=O)cc1OC ; O=C1c2ccc(-c3ccc(O)c(O)c3)cc2C(=O)c2c(O)ccc(O)c21]

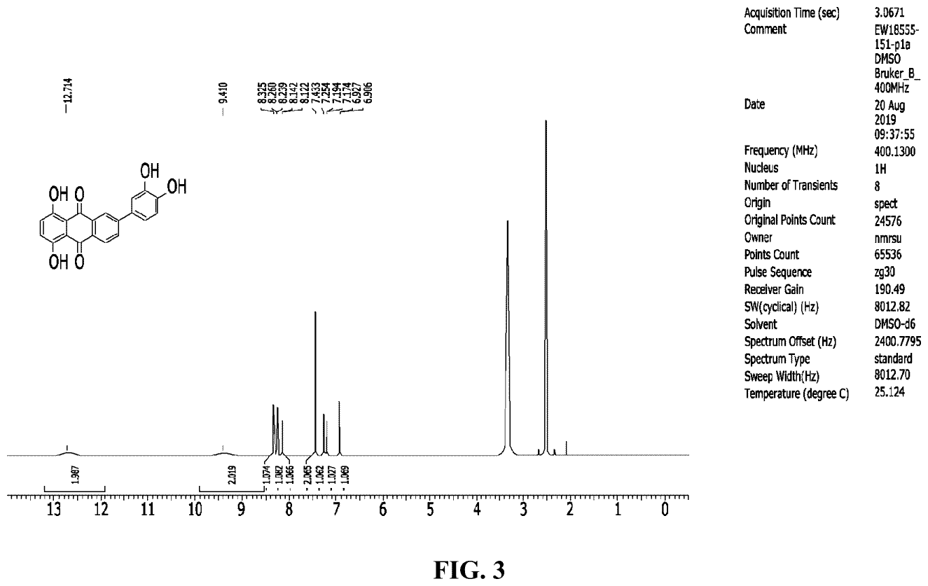

FIG. 3 is a hydrogen atom nuclear magnetic resonance (1H NMR) spectrum of a quinizarin derivative prepared in Example 2 of the present disclosure.

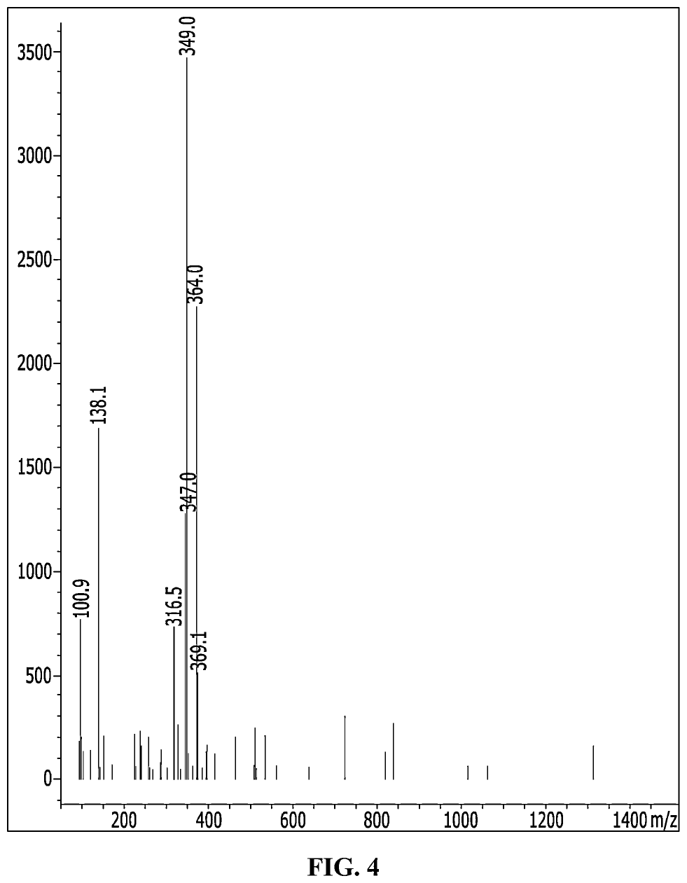

FIG. 4 is an LC-MS result of the quinizarin derivative prepared in Example 2 of the present disclosure.

Through FIGS. 3 and 4, it was confirmed that Compound 2 below was actually prepared.

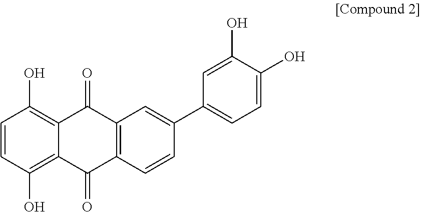 [SMILES: O=C1c2ccc(-c3ccc(O)c(O)c3)cc2C(=O)c2c(O)ccc(O)c21]

### Example 3

A novel quinizarin derivative according to the first aspect of the present disclosure was synthesized according to Reaction Formula 3 below.

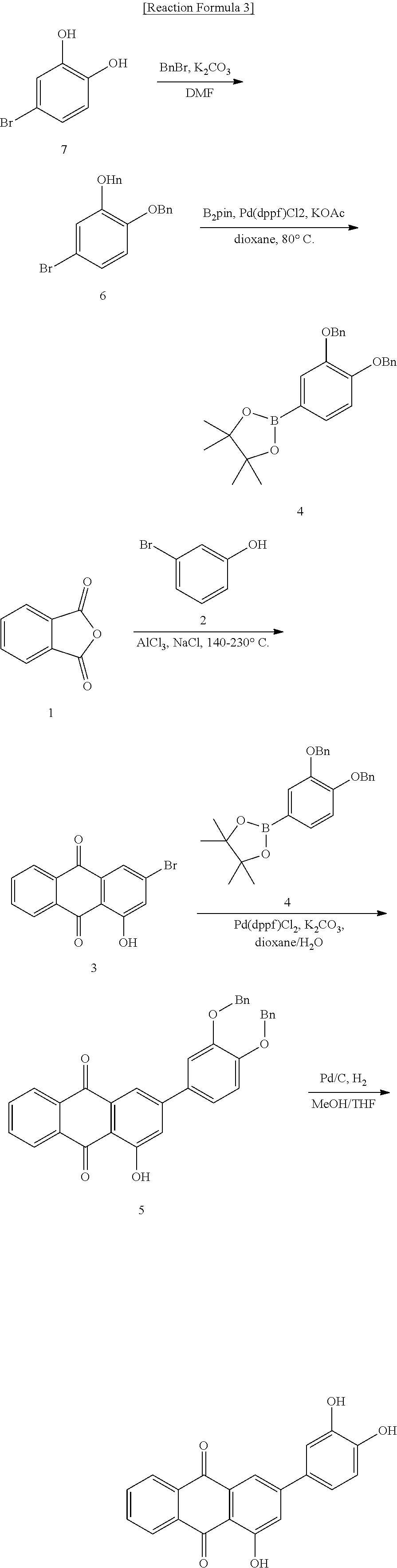

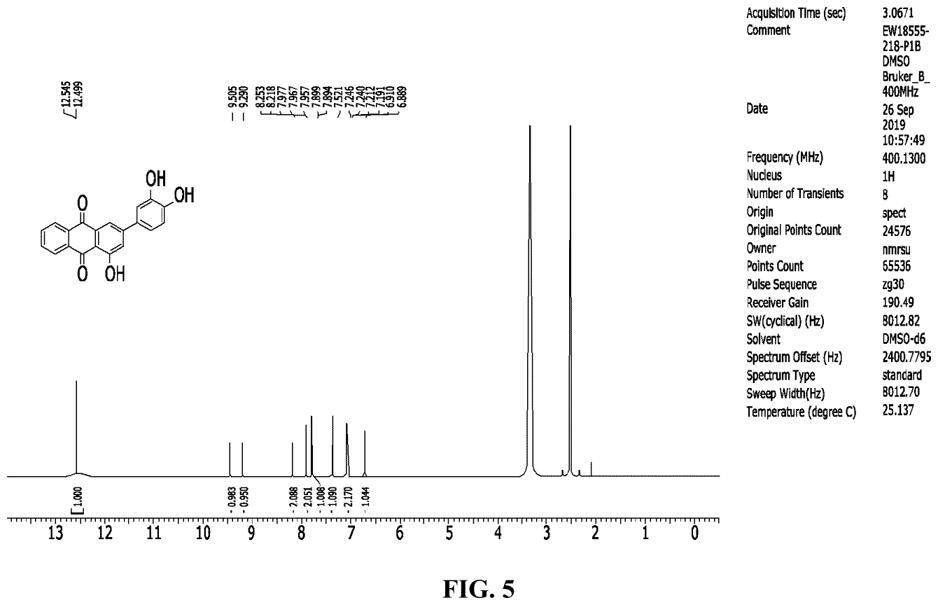

FIG. 5 is a hydrogen atom nuclear magnetic resonance (1H NMR) spectrum of a quinizarin derivative prepared in Example 3 of the present disclosure.

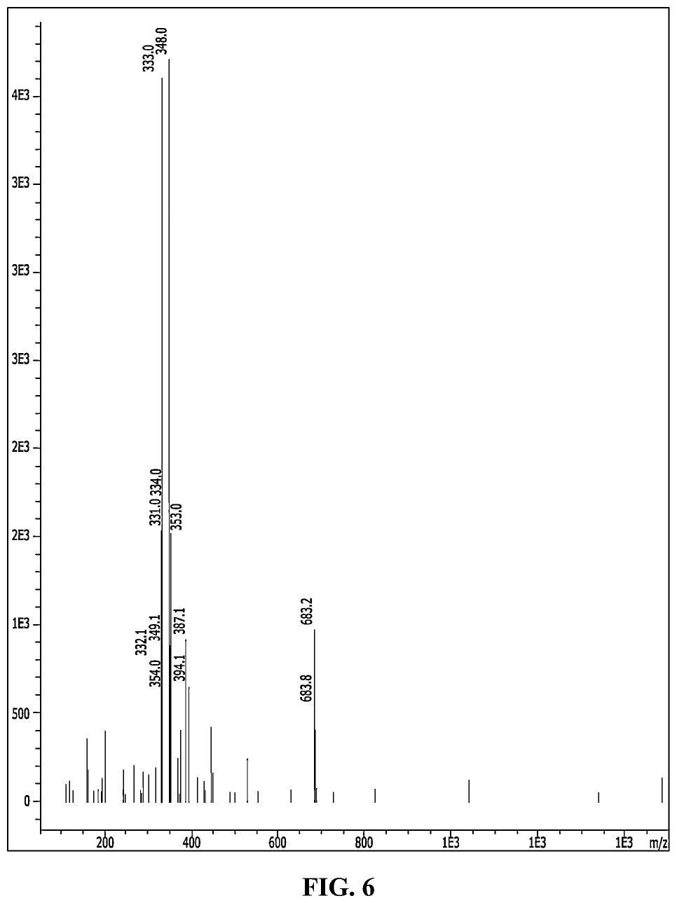

FIG. 6 is an LC-MS result of the quinizarin derivative prepared in Example 3 of the present disclosure.

Through FIGS. 5 and 6, it was confirmed that Compound 3 below was actually prepared.

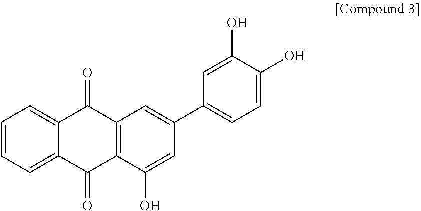 [SMILES: O=C1c2ccccc2C(=O)c2c(O)cc(-c3ccc(O)c(O)c3)cc21]

### Example 4

A novel quinizarin derivative according to the first aspect of the present disclosure was synthesized according to Reaction Formula 4 below.

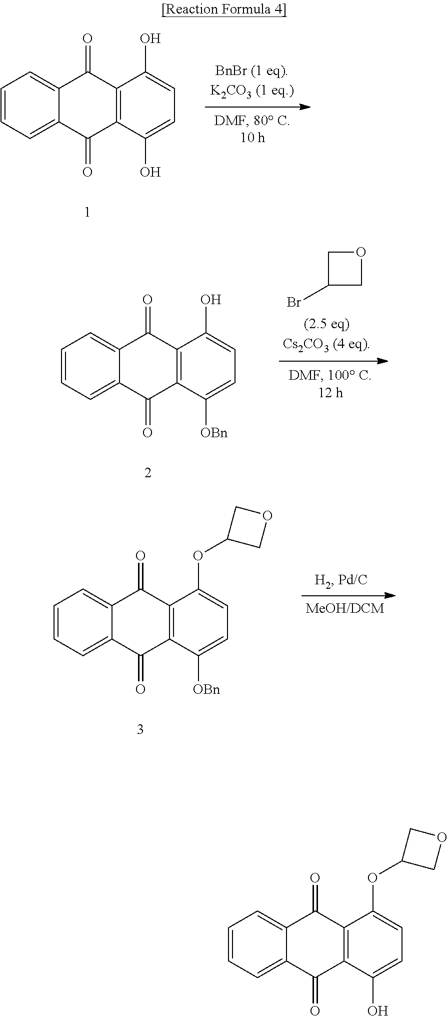 [SMILES: O=C1c2ccccc2C(=O)c2c(O)ccc(O)c21 ; O=C1c2ccccc2C(=O)c2c(OCc3ccccc3)ccc(O)c21 ; BrC1COC1 ; O=C1c2ccccc2C(=O)c2c(OC3COC3)ccc(OCc3ccccc3)c21 ; O=C1c2ccccc2C(=O)c2c(OC3COC3)ccc(O)c21]

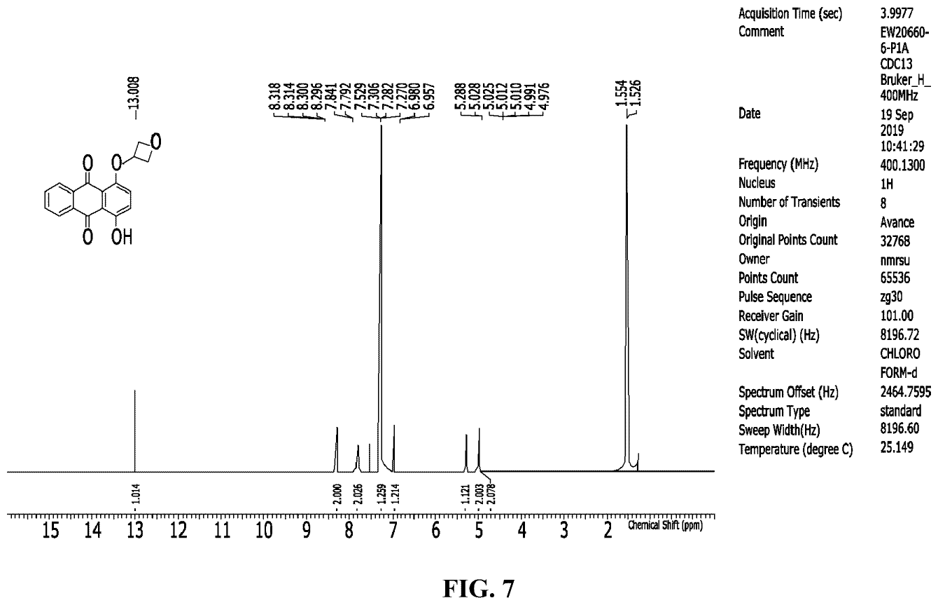

FIG. 7 is a hydrogen atom nuclear magnetic resonance (1H NMR) spectrum of a quinizarin derivative prepared in Example 4 of the present disclosure.

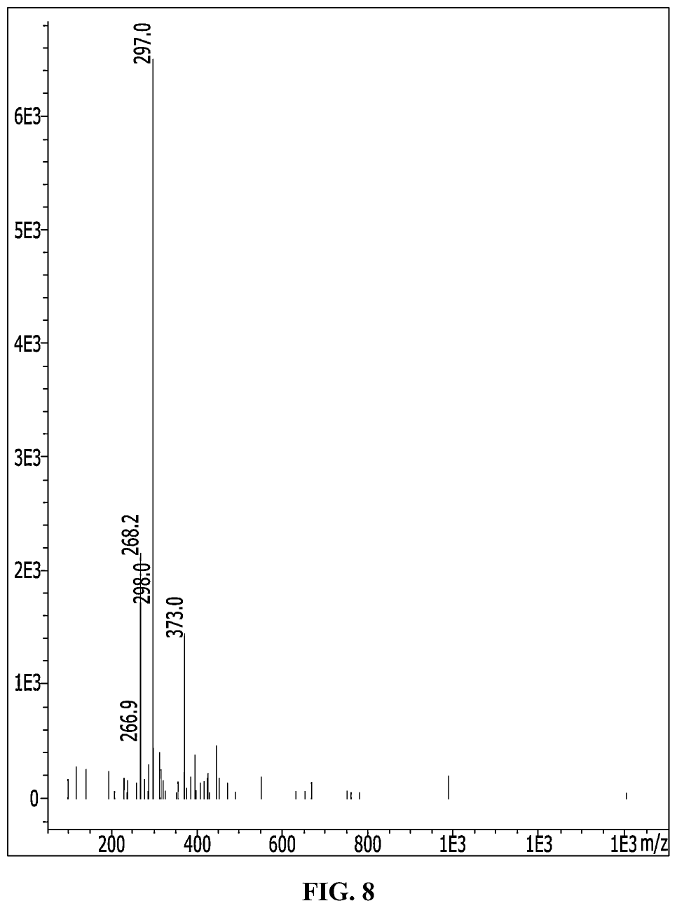

FIG. 8 is an LC-MS result of the quinizarin derivative prepared in Example 4 of the present disclosure.

Through FIGS. 7 and 8, it was confirmed that Compound 4 below was actually prepared.

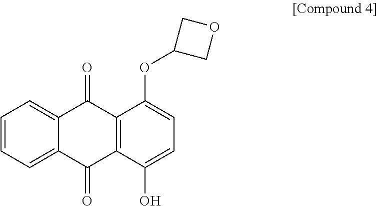 [SMILES: O=C1c2ccccc2C(=O)c2c(OC3COC3)ccc(O)c21]

### Example 5

A novel quinizarin derivative according to the first aspect of the present disclosure was synthesized according to Reaction Formula 5 below.

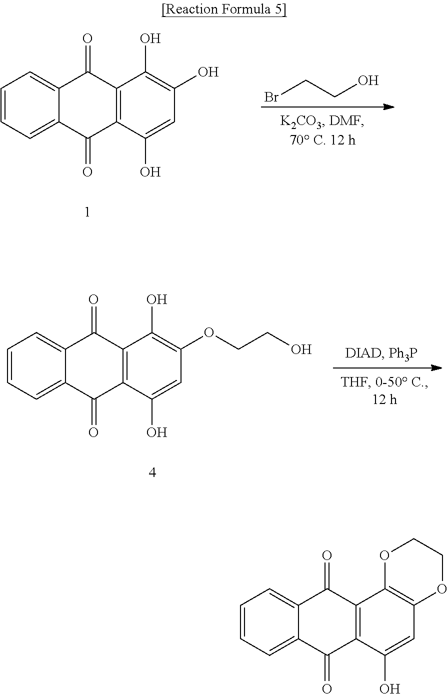

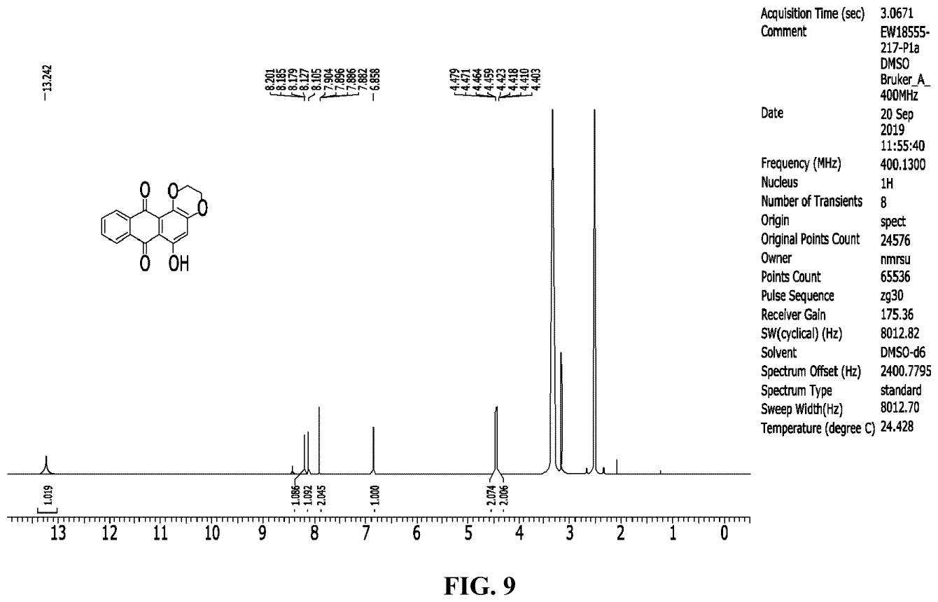

FIG. 9 is a hydrogen atom nuclear magnetic resonance (1H NMR) spectrum of a quinizarin derivative prepared in Example 5 of the present disclosure.

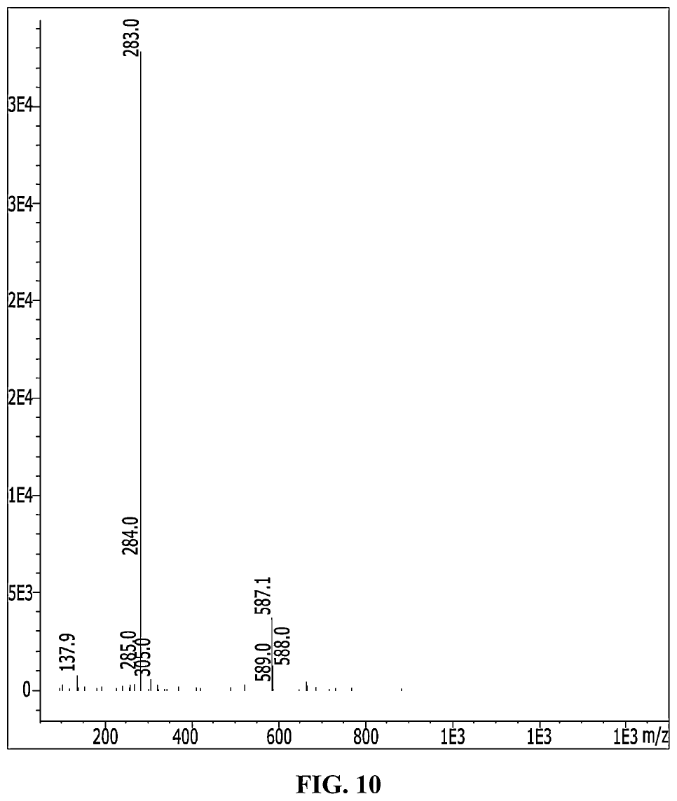

FIG. 10 is an LC-MS result of the quinizarin derivative prepared in Example 5 of the present disclosure.

Through FIGS. 9 and 10, it was confirmed that Compound 5 below was actually prepared.

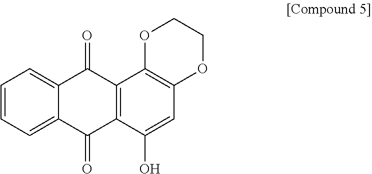 [SMILES: O=C1c2ccccc2C(=O)c2c3c(cc(O)c21)OCCO3]

### [Experimental Example 1] Binding Inhibition Ability Test for BRD2 Protein

To confirm a binding inhibitory effect of a quinizarin derivative of the present disclosure on a BRD2 protein among BET proteins, the following experiment was performed.

First, a compound was diluted in 1:5 subdilution in an assay buffer from a 10 mM stock in dimethyl sulfoxide (DMSO) in a white Optiplate.

Then, a mixture of 100 nM GST BRD2 (BD1, BD2, BD1+BD2) and 100 nM biotinylated acetyl histone H4 (Lys5, 8, 12, 16) peptides was added to the diluent, and then each sample was shaking-cultured in the dark at 300 rpm for 30 minutes at room temperature.

Thereafter, signals were measured with a PerkinElmer Envision HTS Multilabel Reader using a PerkinElmer Alpha Screen protocol. Determination of IC50 values was performed using GraphPad Prism 3.03 software, and the results were shown in Table 1 below.

TABLE 1

| | Compound | BRD2(BD2) |
|---|---|---|
| Example 1 | BBC0503 | 1,737 |
| Example 2 | BBC0504 | 4,437 |
| Example 3 | BBC0506 | 1,748 |
| Example 4 | BBC0507 | 3,120 |
| Example 5 | BBC0510 | 2,728 |

Referring to Table 1, it was confirmed that the quinizarin derivatives according to Examples of the present disclosure all had IC50 values for BRD2 of 5,000 or less, showing excellent inhibitory activity.

### [Experimental Example 2] Experiment for Confirming Lipogenesis Inhibition Ability In Vitro

HepG2 cell lines, human liver epithelial cells, were incubated, pretreated with quinizarin and a substance of Example 2 (BBC0504) at concentrations of 1 μM to 10 μM, and treated with 100 μM palmitic acid (PA) for 24 hours to induce fatty liver at a cellular level. After 24 hours, the cells in each sample were immobilized for 1 hour with a phosphate-buffered salt solution containing 7% formaldehyde, washed with PBS, and stained with a 99% isopropanol solution containing 1% Oil red O for 10 minutes. The differentiation degree of adipocytes was measured by dissolving the stained Oil red O in isopropanol and measuring the absorbance at 510 nm.

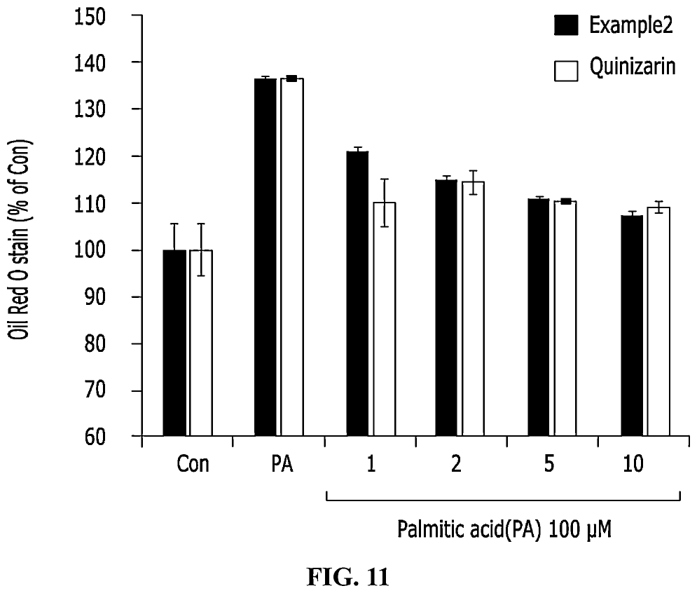

FIG. 11 illustrates results of measuring ability to inhibit lipogenesis of a quinizarin derivative according to an embodiment of the present disclosure.

Referring to FIG. 11, HepG2 cells, which are human liver epithelial cells, were treated with palmitic acid to induce fatty liver, and the ability to inhibit lipogenesis was confirmed at each concentration in Example 2. As a result, it was confirmed that the lipogenesis amount in Example 2 was reduced in a concentration-dependent manner.

### [Experimental Example 3] Experiment for Confirming Ability to Inhibit Expression of Liposynthesis Factors In Vitro

HepG2 cell lines, human liver epithelial cells, were incubated, pretreated with quinizarin and a substance of Example 2 (BBC0504) at concentrations of 1 μM to 10 μM, and treated with 100 μM palmitic acid (PA) for 24 hours to induce fatty liver at a cellular level. After 24 hours, the expression of lipogenesis-related genes SREBP-1 and FAS in cells in each sample was confirmed using RT-PCR. Each sample was added with a trizol reagent to extract total RNA, and the concentration was confirmed using a spectrophotometer. To synthesize cDNA for each gene, RT-PCR reaction was performed using primers targeting the SREBP-1 and FAS genes. β-actin was used as a control group, and after the reaction was completed, the detected cDNA product was confirmed by electrophoresis on a 1.0% agarose gel.

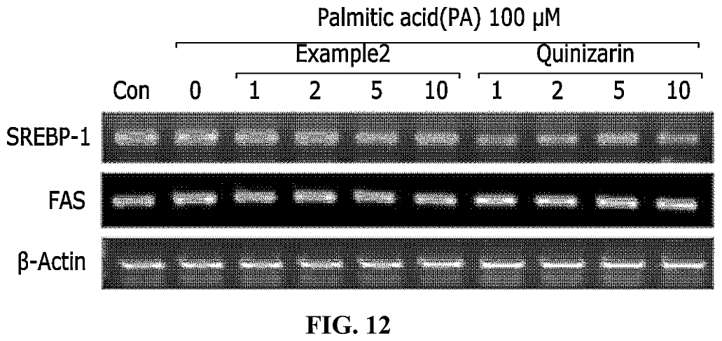

FIG. 12 is a result of RT-PCT reaction to confirm ability to inhibit expression of liposynthesis factors of a quinizarin derivative according to an embodiment of the present disclosure.

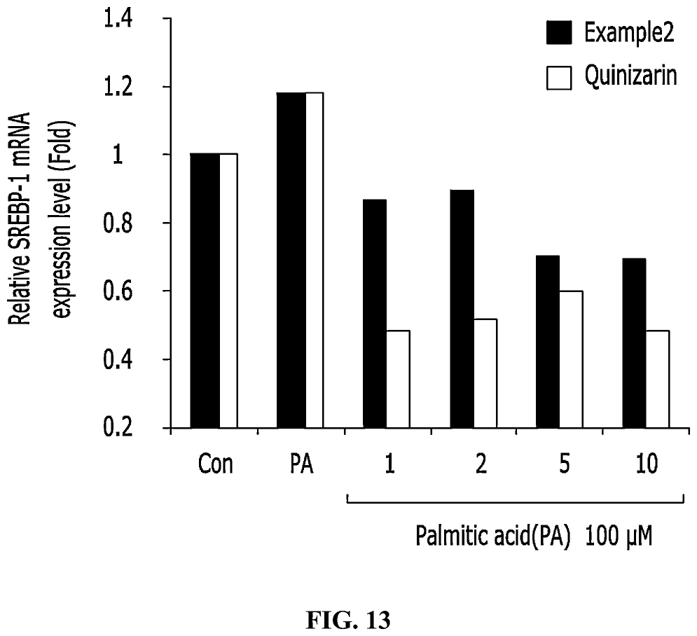

FIG. 13 illustrates a result of confirming SREBP-1 expression after treating fatty liver-induced cells for each concentration with a quinizarin derivative according to an embodiment of the present disclosure.

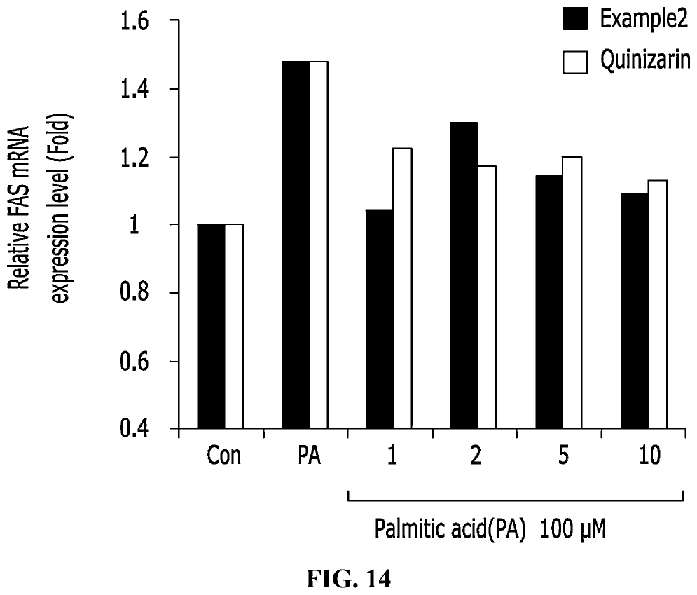

FIG. 14 illustrates a result of confirming FAS expression after treating fatty liver-induced cells with a quinizarin derivative at each concentration according to an embodiment of the present disclosure.

Referring to FIGS. 12 to 14, it was confirmed that gene expression was reduced depending on a concentration of Example 2, and as a result, it was confirmed that the quinizarin derivatives according to Examples showed a protective effect on non-alcoholic fatty liver disease by inhibiting a liposynthesis pathway.

The aforementioned description of the present disclosure is to be exemplified, and it will be understood by those skilled in the art that the present disclosure can be easily modified in other detailed forms without changing the technical spirit or required features of the present disclosure. Therefore, it should be appreciated that the embodiments described above are illustrative in all aspects and are not restricted. For example, each component described as a single form may be implemented in a distributed manner, and similarly, components described as distributed may also be implemented in a combined form.

The scope of the present disclosure is represented by appended claims to be described below rather than the detailed description, and it is to be interpreted that the meaning and scope of the claims and all the changes or modified forms derived from the equivalents thereof come within the scope of the present disclosure.

1. A novel quinizarin derivative, comprising a compound represented by the following Chemical Formula 1 or 2: 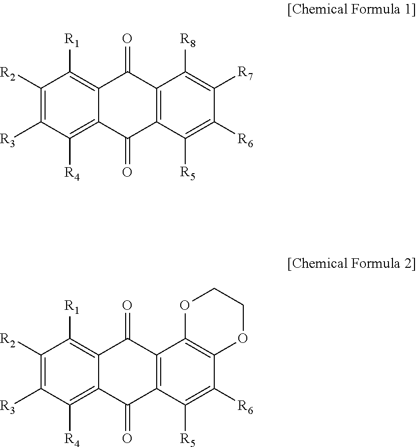
(in Chemical Formulas 1 and 2,
R₁, R₂, R₃, R₄, R₅, R₆, R₇ and R₅ are each independently hydrogen, hydroxy, linear or branched C₁-C₁₂ alkyl which may be substituted, C₃-C₁₂ cycloalkyl which may be substituted, C₆-C₁₈ aryl which may be substituted, or alkoxy, and
the substitution is performed by oxygen, nitrogen, sulfur, hydroxyl, linear or branched C₁-C₆ alkyl, C₆-C₂₀ aryl, halogen, alkoxy, ether or a combination thereof).

2. The novel quinizarin derivative of claim 1, wherein the quinizarin derivative includes any one of the following compounds:

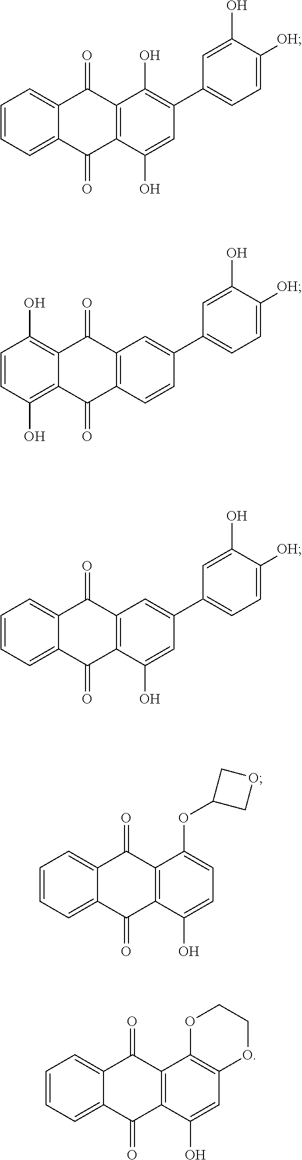 [SMILES: O=C1c2ccccc2C(=O)c2c(O)c(-c3ccc(O)c(O)c3)cc(O)c21 ; O=C1c2ccc(-c3ccc(O)c(O)c3)cc2C(=O)c2c(O)ccc(O)c21 ; O=C1c2ccccc2C(=O)c2c(O)cc(-c3ccc(O)c(O)c3)cc21 ; O=C1c2ccccc2C(=O)c2c(OC3COC3)ccc(O)c21 ; O=C1c2ccccc2C(=O)c2c3c(cc(O)c21)OCCO3]

3. A method for preparing the novel quinizarin derivative according to claim 1, comprising reacting an anthracene derivative with a compound represented by Chemical Formula 3, 4, or 5 below: 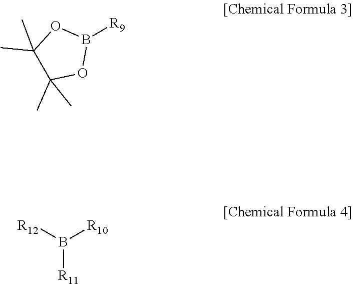
Br—R₁₃ [Chemical Formula 5]
(in Chemical Formulas 3 to 5,
R₉, R₁₀, R₁₁, R₁₂, and R₁₃ are each independently hydrogen, hydroxy, linear or branched C₁-C₁₂ alkyl which may be substituted, C₃-C₁₂ cycloalkyl which may be substituted, C₆-C₁₈ aryl which may be substituted, or alkoxy, and
the substitution is performed by oxygen, nitrogen, sulfur, hydroxyl, linear or branched C₁-C₆ alkyl, C₆-C₂₀ aryl, halogen, alkoxy, ether or a combination thereof).

4. The method for preparing the novel quinizarin derivative of claim 3, wherein the anthracene derivative is selected from the group consisting of 2-bromo-1,4-dihydroxy-anthracene-9,10-dione, 6-bromo-1,4-dihydroxy-anthracene-9,10-dione, 3-bromo-1-hydroxy-anthracene-9,10-dione, 1,4-dihydroxy-anthracene-9,10-dione, 1,2,4-trihydroxy-anthracene-9,10-dione, and combinations thereof.

5. The method for preparing the novel quinizarin derivative of claim 3, wherein the compound represented by Chemical Formula 3 includes 2-(3,4-dimethoxyphenyl)-4,4,5,5-tetramethyl-1,3,2-dioxaborolane, or 4,4,5,5-tetramethyl-2-(4,4,5,5-tetramethyl-1,3,2-dioxaborolan-2-yl)-1,3,2-dioxaborolane.

6. The method for preparing the novel quinizarin derivative of claim 3, wherein the compound represented by Chemical Formula 4 includes 3,4-dimethoxyphenylboronic acid.

7. The method for preparing the novel quinizarin derivative of claim 3, wherein the compound represented by Chemical Formula 5 includes benzyl bromide or 2-bromoethanol.

8. The method for preparing the novel quinizarin derivative of claim 3, wherein the quinizarin derivative includes any one of the following compounds:

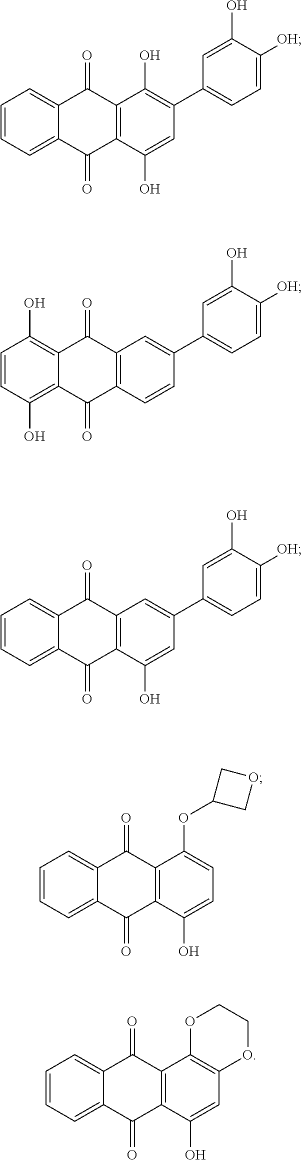 [SMILES: O=C1c2ccccc2C(=O)c2c(O)c(-c3ccc(O)c(O)c3)cc(O)c21 ; O=C1c2ccc(-c3ccc(O)c(O)c3)cc2C(=O)c2c(O)ccc(O)c21 ; O=C1c2ccccc2C(=O)c2c(O)cc(-c3ccc(O)c(O)c3)cc21 ; O=C1c2ccccc2C(=O)c2c(OC3COC3)ccc(O)c21 ; O=C1c2ccccc2C(=O)c2c3c(cc(O)c21)OCCO3]

9. A bromodomain extra-terminal (BET) protein inhibitor comprising the novel quinizarin derivative according to claim 1.

10. A composition for treating non-alcoholic fatty liver comprising the novel quinizarin derivative according to claim 1.
